(19) United States
(12) Patent Application Publication
(10) Pub. No.: US 2026/0174793 A1
(43) Pub. Date: Jun. 25, 2026

(54) COSMETIC OR PHARMACEUTICAL PREPARATION FOR SKIN CARE AND IMPROVING SKIN CONDITION

(71) Applicant: MERRYVITAL AG, Bottighofen (CH)

(72) Inventor: Christian BENZING, Stuttgart (DE)

(73) Assignee: MERRYVITAL AG, Bottighofen (CH)

(21) Appl. No.: 18/712,662

(22) PCT Filed: Nov. 23, 2021
(86) PCT No.: PCT/IB2021/060868

§ 371 Date: May 22, 2024

(51) Int. Cl. A61K 31/728; A61K 8/9789; A61K 36/28; A61K 38/00; A61Q 19/00

(52) U.S. Cl. CPC A61K 31/728; A61K 8/9789; A61K 36/28; A61K 38/00; A61Q 19/00

(57) ABSTRACT

The present invention relates to a topically applicable cosmetic or pharmaceutical preparation for skin care and for improving the condition of the skin.

The present invention relates to a topically applicable cosmetic or pharmaceutical preparation for skin care and for improving the condition of the skin.

Skin care is part of body care and, in a broader sense, includes the cleansing and treatment of the skin with the aim of maintaining the skin's natural protective mechanisms and health and improving general well-being. Skin care products are cosmetic or medical preparations for the care, firming and moisturizing of stressed skin and for the after-treatment or prophylaxis of acute dermatoses. Commonly used active ingredients include, for example, urea, dexpanthenol, allantoin and various oils and fats.

What all known skin care products have in common, however, is that they do not always work reliably and do not work for all those affected. In addition, intolerances and allergies can develop, which makes the use of alternative products necessary. There is therefore still a significant need for new skin care products, especially those with firming and anti-cellulite effects.

The underlying object of the present invention is therefore to provide new skin care products which can bring about an advantageous tightening of sagging skin.

A further object of the present invention is to provide new skin care products for the cosmetic prophylaxis and cosmetic treatment of cellulite.

The underlying object of the present invention is therefore to provide new skin care products which avoid the disadvantages of the prior art.

This object is achieved according to the invention by a topically applicable cosmetic or pharmaceutical preparation for skin care and for improving the condition of the skin, which contains a combination of at least 3 raw materials selected from
a) very low molecular weight hyaluronic acid with a molecular weight of less than 10 Daltons,
b) Nara oil,
c) Green Caviar Extract,
d) Dipeptide Diaminobutyroyl Benzylamide Diacetate (Syn-Ake),
e) Coenochloris Signiensis Extract (Snow Algae Extract),
f) Zimtextrakt (Cinnamomum cassia Bark Extract),
g) Plankton Extract,
h) Acetyl Cyclohexapeptide-34,
i) Vanillyl Butyl Ether,
j) Nicotinate or chili extract,
k) Peptides,
l) Hydroxystilbene or plant extracts containing hydroxystilbene and
m) Glacier water

Particularly preferred according to the invention are the following combinations, which are particularly suitable for the stated cosmetic purposes:

### Day Cream:

Cinnamon extract
Kahai oil
Pinolumin (Pinus cembra Wood Extract)
Active ingredient booster: Palmitoyl Tripeptide-38

### Night Cream:

Cinnamon extract
Derma membrane structures (DMS), in particular SLM 2026, available from Lipoid Kosmetik AG, CH-6312 Steinhausen, Switzerland.
Pinolumin (Pinus cembra Wood Extract)
Active ingredient booster: Acetyl Octapeptide-3

### Eye Cream:

Cinnamon extract
Argan oil butter (Hydrogenated Argan Butter)
Pinolumin (Pinus cembra Wood Extract)
Active ingredient booster: low molecular weight hyaluronic acid <10 Da

### Facial Serum:

Cinnamon extract
Vitamin C Ester (3-O-Ethyl Ascorbic Acid)
Pinolumin (Pinus cembra Wood Extract)
Active ingredient booster: Pullulan

### Platinum Facial Serum

Cinnamon extract
Acetyl Tetrapeptide-17, Collodial Platinum
Acetyl Octapeptide-3
Active ingredient booster: Truffle Extract (Tuber Magnatum Extract)

### SPF 30 Face Cream

Cinnamon extract
Truffle Extract (Tuber Magnatum Extract)
Hormone-free Sun Protection Factor (SPF) system (without suspected hormonal UV filters:
Cinnamic acid esters, salicylates, octocrylene)
Active ingredient booster: Acetyl Octapeptide-3

### Luxury Face Cream:

Nara oil
Cinnamon extract
Acetyl sh-Pentapeptide-35, Diamond Powder
Active ingredient booster: Schneealgenextrakt (Coenochloris Signiensis Extract)

### Anti-Cellulite Gel-Sensitive:

Plankton extract,
Cinnamon extract
Acetyl Cyclohexapeptide-34
Active ingredient booster: Vanillyl Butyl Ether

### Anti-Cellulite Gel: —Performance:

Acetyl Octapeptide-3
Cinnamon extract
Acetyl Cyclohexapeptide-34
Active ingredient booster: Benzylnicotinate

In the context of the present invention, the aforementioned combinations of three active ingredients in combination with the respective active ingredient boosters are particularly preferred.

For all of the relative or percentage weight-related quantities mentioned below, it should be noted that these must be selected within the scope of the compositions according to the invention in such a way that in total they always add up to 100% or 100% by weight in the composition according to the invention, including all components or ingredients or additives or constituents or excipients. But this goes without saying for the person skilled in the art.

Furthermore, the person skilled in the art can deviate from the numerical, range, weight and quantity information given below, depending on the application or on a case-by-case basis, without departing from the scope of the present invention.

It also goes without saying that refinements, embodiments, advantages and the like, which are listed below only for one aspect of the invention in order to avoid repetition, also apply accordingly with regard to the other aspects of the invention.

A preferred preparation according to the invention contains the ingredients mentioned in claim 1 in an effective, in particular in a cosmetically and/or pharmaceutically effective amount.

Very low molecular weight hyaluronic acid with a molecular weight of less than 10 Daltons is able to be absorbed into the skin. It ensures that water is stored in the connective tissue of the skin. The additional moisture not only makes the skin plumper and firmer, but wrinkles are also visibly reduced. When using low molecular weight hyaluronic acid, moisture is stored sustainably in the skin. It therefore has more advantages and, above all, a more lasting effect compared to higher molecular hyaluronic acid types.

A preparation according to the invention preferably contains a total content of very low molecular weight hyaluronic acid with a molecular weight of less than 10 Daltons of 0.05 to 5% by weight, in particular 0.5 to 2% by weight, particularly preferably 0.75 to 1.25% by weight.

Nara oil (INCI: Acanthosicyos horridus Seed Oil) is obtained from the Nara pumpkin plant, which belongs to the genus Acanthosicyos and is endemic to Namibia. Nara oil is rich in unsaturated (omega-6) fatty acids. The proportion of linoleic acid is around 55 percent. Linoleic acid is very important for the skin's water balance and a well-functioning skin barrier. Nara oil also contains a lot of vitamin E.

A preparation according to the invention preferably contains a total Nara oil content of 2 to 10% by weight, in particular 3 to 8% by weight, particularly preferably 4 to 6% by weight.

Green Caviar Extract (INCI: Caulerpa Lentillifera Extract) contains high levels of amino acids such as threonine, valine, aspartic acid, glutamic acid, etc. Some amino acids (glutamic acid, proline, glycine) act like collagen, which is effective for skin elasticity. These types of amino acids prevent water loss and help absorb moisture; they also improve the skin's immune system and maintain skin elasticity. Green Caviar Extract is said to have a blood purifying effect and inhibit ROS and lipid peroxides (antioxidant activity). Green Caviar Extract also exhibits heavy metal chelating activity.

A preparation according to the invention preferably contains a total green caviar extract content of 1 to 10% by weight, in particular 2.5 to 7.5% by weight, particularly preferably 3 to 5% by weight.

Dipeptide Diaminobutyroyl Benzylamide Diacetate (Syn-Ake) is a tripeptide with a molecular weight of less than 500 Daltons, meaning it is small enough to penetrate well and easily into the skin. Syn-Ake is said to be able to smooth small skin wrinkles that arise from facial movements, such as smile lines or crow's feet.

A preparation according to the invention preferably contains a total dipeptide diaminobutyroyl benzylamide diacetate content of 1 to 10% by weight, in particular 1 to 5% by weight, particularly preferably 2 to 3% by weight.

Coenochloris Signiensis Extract (Snow Algae Extract) is intended to mimic the mechanism of calorie restriction and increase the longevity of skin cells. Coenochloris Signiensis is an extremophile algae that has managed to develop survival strategies that allow it to grow on glaciers and in permanent snow.

The extract of this algae is said to activate two key factors of the calorie restriction pathway at the cellular level: the Klotho longevity gene and the AMPK energy sensor, which together lead to improved cellular defense, resistance to oxidative stress, cell detoxification and repair.

The results in the skin are the renewed production of collagen and a rejuvenation of the dermal-epidermal interface. As a result, the skin barrier is strengthened and the skin appears fresher and detoxified as age spots are less visible.

A preparation according to the invention preferably contains a total content of Coenochloris Signiensis Extract of 0.5 to 10% by weight, in particular 1 to 5% by weight, particularly preferably 2 to 3% by weight.

Cinnamon extract (Cinnamomum Cassia Bark Extract) is mostly used in cosmetics as a fragrance. According to the invention, however, it is used here as a preservative in order to be able to reduce or completely eliminate more aggressive preservatives (parabens, phenoxyethanol, DMDM hydantoin, etc.).

A preparation according to the invention preferably contains a total cinnamon extract content of 0.05 to 2.5% by weight, in particular 0.5 to 1.5% by weight, particularly preferably 0.1 to 0.5% by weight.

Plankton extract is a marine polysaccharide made from micro-organisms called plankton.

Plankton float in the sea in very large quantities and use carbon dioxide and sunlight to produce the energy they need for their metabolism and growth. Because plankton are constantly exposed to massive amounts of UV radiation, they have developed a special enzyme that repairs cell damage caused by intense sunlight. However, the vital substances in plankton not only protect the skin from UV-related oxidation and premature skin aging, but also strengthen the skin's protective barrier. In addition, the ingredients in plankton are said to accelerate the differentiation of skin cells and thus give the skin more freshness and youthfulness.

A preparation according to the invention preferably contains a total plankton extract content of 0.05 to 5% by weight, in particular 0.1 to 2.5% by weight, particularly preferably 1 to 2% by weight.

Acetyl Cyclohexapeptide-34 is an anti-cellulite active ingredient that is sold under the trade name Liposuctionine by the company Infinitec Activos or by Infinitec under the trade name X50 Silhouette as a mixture with stabilizers.

A preparation according to the invention preferably contains a total acetylcyclohexapeptide-34 content of 0.00005 to 1% by weight, in particular 0.0005 to 0.5% by weight, particularly preferably 0.005 to 0.01% by weight.

Vanillyl butyl ether is an ether of monohydroxybenzoic acid. It is added to foods as a flavoring agent. It has a distinctive burning hot pepper character and can be used in spice flavors such as pepper, cinnamon and ginger. It is also included in cosmetics and personal care products as a fragrance, oral care agent, hair conditioning agent, and warming or cooling agent. Vanilloids contain vanillyl groups that bind to the transient receptor potential type V1 channel (vanilloid receptor 1, TRPV1), which responds to noxious stimuli such as high temperatures and acidic pH.

This causes the neurons to release glutamate, adenosine triphosphate (ATP), and a variety of neuropeptides, resulting in the feeling of warmth.

A preparation according to the invention preferably contains a total vanillyl butyl ether content of 0.05 to 5% by weight, in particular 0.1 to 2.5% by weight, particularly preferably 0.25 to 1.25% by weight.

Nicotinates, in particular methyl nicotinate and propyl nicotinate, are known from the prior art as blood circulation-promoting and warming agents that are applied topically. They are often used alternatively to or together with chili extract.

A preparation according to the invention preferably contains a total nicotinate content of 0.05 to 5% by weight, in particular 0.1 to 2.5% by weight, particularly preferably 0.25 to 1.25% by weight.

A preparation according to the invention preferably contains a total chili extract content of 0.05 to 10% by weight, in particular 0.1 to 5% by weight, particularly preferably 0.25 to 2.5% by weight.

A preparation according to the invention preferably contains a total peptide content of 0.0001 to 5% by weight, in particular 0.01 to 4% by weight, preferably 0.1 to 3% by weight, particularly preferably 0.5 to 2% by weight.

Peptides that are particularly suitable according to the invention are selected from the tripeptides Gly-His-Lys, Lys-Val-Lys, Lys-Val-Dab, Lys-Phe-Lys, Lys-Ile-Lys, Dab-Val-Lys, Lys-Val-Orn, Lys-Val-Dap, Dap-Val-Lys and Gly-His-Arg, —the tetrapeptides Gly-Gln-Pro-Arg (Rigin), Gly-Gln-Arg-Pro, Val-Val-Arg-Pro, Rigin-Ana-Ioga and ALAMCAT tetrapeptides, —the pentapeptide Lys-Thr-Thr-Lys-Ser, —the hexapeptides Val-Gly-Val-Ala-Pro-Gly, Ala-Asp-Leu-Lys-Pro-Thr (Hexapeptide-3), Hexapeptide-4, Hexapeptide-5, Hexapeptide-6, Hexapeptide-8, Hexapeptide-9 and Hexapeptide-10, —the derivatives of the tri-, tetra-, penta- and hexapeptides mentioned, which are N-acylated and/or esterified with at least one, preferably linear, C2-C22 fatty acid, in particular N-Palmitoyl-Gly-His-Lys, N-Palmitoyl-Lys-Val-Lys, N-Myristoyl-Gly-His-Arg, N-Palmitoyl-Gly-Gln-Pro-Arg, N-Palmitoyl-Lys-Thr-Thr-Lys-Ser, N-Palmitoyl-Val-Gly-Val-Ala-Pro-Gly, Acetyl-Hexapeptide-3, Myristoyl Hexapeptide-5, Myristoyl Hexapeptide-8 as well as mixtures of N-Palmitoyl-Gly-His-Lys and N-Palmitoyl-Gly-Gln-Pro-Arg.

Preferred peptides within the meaning of the present invention are also the peptides disclosed in WO/2010/082175, to which reference is hereby made in its entirety.

According to the invention, it may be preferred that the peptides used as active ingredients are N-acylated and/or esterified with at least one, preferably linear, C2-C22 fatty acid to improve penetration into the skin. C8-C18 fatty acids are particularly preferred for N-acylation and/or esterification; myristic acid (C14) and palmitic acid (C16) are very particularly preferred. Substitution of the peptides with a benzyloxycarbonyl group on the terminal amino group is also preferred.

A preferred peptide according to the invention is the tripeptide Gly-His-Lys, which, for example, is available under the name “Omega-CH Activator” from GfN or in acylated form (N-Palmitoyl-Gly-His-Lys) under the name Biopeptide CL from Sederma, but (in acylated form) also a component of the product Matrixyl 3000 from Sederma. The tripeptide Gly-His-Lys can also be used as a copper salt (Cu2+) and is available as such from ProCyte Corporation. Furthermore, analogues of Gly-His-Lys can preferably be used, with a maximum of two amino acids being substituted by suitable other amino acids. According to the invention, Ala, Leu and Ile are preferably suitable for substituting Gly. The amino acids which are particularly preferred according to the invention and which can replace His or Lys contain a side chain with a nitrogen atom which is predominantly charged at pH 6, e.g. Pro, Lys, Arg, His, desmosine and isodesmosine.

Lys is particularly preferably replaced by Arg, Orn or citrulline. Another peptide preferred according to the invention is Gly-His-Arg (INCI name: Tripeptide-3) and its derivative N-Myristoyl-Gly-His-Arg, which, for example, is available under the name Collasyn 314-GR from Therapeutic Peptide Inc.

Further tripeptides preferred according to the invention are selected from Lys-Val-Lys, Lys-Val-Dab (Dab=diaminobutyric acid), Lys-Phe-Lys, Lys-Ile-Lys, Dab-Val-Lys, Lys-Val-Orn, Lys-Val-Dap (Dap=diaminopropionic acid) and Dap-Val-Lys. A likewise preferred tripeptide is Palmitoyl-Lys-Val-Lys, for example, available from Pentapharm under the name SYN®-COLL.

Further tetrapeptides preferred according to the invention are the tetrapeptide Rigin and Rigin-based tetrapeptides as well as ALAMCAT tetrapeptides. Rigin has the sequence Gly-Gln-Pro-Arg. Rigin-based tetrapeptides include the Rigin analogs and Rigin derivatives, in particular N-Palmitoyl-Gly-Gln-Pro-Arg (INCI name: Palmitoyl Tetrapeptide-1), which, for example, is available under the name Eyeliss from Sederma, but is also a component of the product Matrixyl 3000 from Sederma. The Rigin analogs include those in which the four amino acids are rearranged and/or in which a maximum of two amino acids are substituted compared to Rigin, e.g. the sequence Ala-Gln-Thr-Arg. Preferably at least one of the amino acids of the sequence has a Pro or Arg and particularly preferably the tetrapeptide includes both Pro and Arg, although their order and position can vary. The substituting amino acids can be selected from any amino acid defined below. Particularly preferred Rigin-based tetrapeptides include: Xaa-Xbb-Arg-Xcc, Xaa-Xbb-Xcc-Pro, Xaa-Xbb-Pro-Arg, Xaa-Xbb-Pro-Xcc, Xaa-Xbb-Xcc-Arg, where Xaa, Xbb and Xcc can be the same or different amino acids and where Xaa is selected from Gly and the amino acids that can substitute Gly, Xbb is selected from Gin and the amino acids that can substitute Gin, Xcc is selected from Pro or Arg and the amino acids that can substitute Pro and Arg. The preferred amino acids that can replace Gly include an aliphatic side chain, e.g. β; -Ala, Ala, Val, Leu, Pro, sarcosine (Sar) and isoleucine (Ile). The preferred amino acids that can replace Gln include a side chain with an amino group that is predominantly uncharged at neutral pH (pH 6-7), e.g. Asn, Lys, Orn, 5-hydroxyproline, citrulline and canavanine. The preferred amino acids that can replace Arg include a side chain with a nitrogen atom that is predominantly charged at pH 6, for example Pro, Lys, His, desmosine and isodesmosine.

According to the invention, Gly-Gln-Arg-Pro and Val-Val-Arg-Pro are preferred as Rigin analogs. ALAMCAT tetrapeptides are tetrapeptides that contain at least one amino acid with an aliphatic side chain, e.g.; β-Ala, Ala, Val, Leu, Pro, sarcosine (Sar) and isoleucine (Ile). Furthermore, ALAMCAT tetrapeptides contain at least one amino acid with a side chain with an amino group that is predominantly uncharged at neutral pH (pH 6-7), e.g. Gln, Asn, Lys, Orn, 5-hydroxyproline, citrulline and canavanine. Furthermore, ALAMCAT tetrapeptides contain at least one amino acid with a side chain with a nitrogen atom that is predominantly charged at pH 6, e.g. Arg, Pro, Lys, His, desmosine and isodesmosine. As a fourth amino acid, ALAMCAT tetrapeptides can contain any amino acid; however, the fourth amino acid is also preferably selected from the three groups mentioned above.

Further peptides preferred according to the invention are the pentapeptide Lys-Thr-Thr-Lys-Ser and its N-acylated derivatives, particularly preferably N-Palmitoyl-Lys-Thr-Thr-Lys-Ser, which is available under the name Matrixyl from Sederma

Further hexapeptides preferred according to the invention are the hexapeptide Val-Gly-Val-Ala-Pro-Gly and its N-acylated derivatives, particularly preferably N-Palmitoyl-Val-Gly-Val-Ala-Pro-Gly, which is available under the name Biopeptide EL from Sederma. Further peptides preferred according to the invention are the hexapeptides and/or their N-acylated derivatives, Ala-Asp-Leu-Lys-Pro-Thr (Hexapeptide-3, e.g. Peptide 02 from Vincience), Acetyl-Hexapeptide-3 (Argireline from Lipotec), Hexapeptide-4 (e.g. Collasyn 6KS from Therapeutic Peptide Inc. (TPI)), Hexapeptide-5 (e.g. Collasyn 6VY from TPI), Myristoyl Hexapeptide-5 (e.g. Collasyn 614VY from TPI), Myristoyl Hexapeptide-6 (e.g. Collasyn 614VG from TPI), Hexapeptide-8 (e.g. Collasyn 6KS from TPI), Myristoyl Hexapeptide-8 (e.g. Collasyn Lipo-6KS from TPI), Hexapeptide-9 (e.g. Collaxyl from Vincience) and Hexapeptide-10 (e.g. Collaxyl from Vincience or Seriseline from Lipotec).

Particularly preferred peptides according to the invention are
Acetyl Tetrapeptide 17, especially in form conjugated to colloidal gold or platinum, available from Infinitec Activos S.L.: Particularly preferred is the maltodextrin-based Acetyl Tetrapeptide 17 powder conjugated to colloidal platinum.
Acetyl Octapeptide-3, available under the name SNAP-8™ from Lipotec S.A.U.
The peptide available under the name Matrixyl® synthe'6 from Sederma.
Acetyl Hexapeptide-8 and Pentapeptide-18, which are available under the name AR-GIRELOX™ from Lipotec S.A.U. as a peptide solution.

The preparation according to the invention can contain one peptide or a plurality of different peptides.

A preparation according to the invention preferably contains a total hydroxystilbene content of 0.001 to 10% by weight, in particular 0.001 to 5% by weight, preferably 0.001 to 4% by weight, particularly preferably 0.005 to 1% by weight, extremely preferably 0.006 to 1% by weight.

Hydroxystilbenes which are particularly suitable according to the invention are selected from hydroxystilbenes, hydroxystilbene oligomers, their alkyl ethers and/or their esters.

Hydroxystilbenes are an extensive group of natural substances, mostly phytoalexins; many of them are obtained from conifers and are often present as glycosides and/or methyl ethers. But they can also be produced synthetically. Hydroxystilbenes esterified with acids, for example with phosphoric acid, are also suitable according to the invention. Most hydroxystilbenes exist in the (E) form, but some also occur in the (Z) form. One of the most important representatives is pinosylvin (3,5-stilbenediol). In the (E) form, pinosylvin forms needles that are insoluble in water and soluble in acetone. (E) and (Z) isomers occur alongside the corresponding mono- and dimethyl ethers in pines, conifers and other softwoods. Pinosylvin has fungicidal and antibacterial properties. Pinosylvin monomethyl ether is known as an antifeedant.

Hydroxystilbenes can oligomerize via the attack of a hydroxyl group on the ethylenic double bond of another hydroxystilbene molecule to form a dihydrofuran ring. Well-known examples of the oligomers of hydroxystilbenes are the dimers of resveratrol, in particular of (E)-resveratrol, the so-called viniferins, in particular the transepsilon-viniferin. Well-known trimers and tetramers of resveratrol are vitisin E and vitisin D. According to the invention, the oligomers of hydroxystilbenes are understood to mean dimers, trimers, tetramers, pentamers and hexamers of hydroxystilbenes.

Hydroxystilbenes particularly preferred according to the invention are 4′-hydroxystilbene, 2′,4′-dihydroxystilbene, 3′,4′-dihydroxystilbene, 4,4′-dihydroxystilbene, 3,5-dihydroxystilbene (pinosylvin), 3-hydroxy-5-methoxystilbene (pinosylvin monomethyl ether), 2′,4′,4-trihydroxystilbene, 3′,4′,4-trihydroxystilbene, 2,4,4′-trihydroxystilbene, 3,4,4′-trihydroxystilbene, 3,4′,5-trihydroxystilbene (resveratrol), 2′,3,4-trihydroxystilbene, 2,3′,4-trihydroxystilbene, 2′,2,4′-trihydroxystilbene, 2,4,4′,5-tetrahydroxystilbene, 2′,3,4′,5-tetrahydroxystilbene, 2,2′,4,4′-tetrahydroxystilbene, 3,3′,4′,5-tetrahydroxystilbene, 2,3′,4,4′-tetrahydroxystilbene, 3,3′,4,4′-tetrahydroxystilbene, 3,3′,4′,5,5′-pentahydroxystilbene, 2,2′,4,4′,6-pentahydroxystilbene, 2,3′,4,4′,6-pentahydroxystilbene, 2,2′,4,4′,6,6′-hexahydroxystilbene, their alkyl ethers and/or their esters as well as trans-epsilon-viniferin, vitisin E and vitisin D and their alkyl ethers and/or their esters.

A particularly preferred hydroxystilbene according to the invention is 3,5-dihydroxystilbene (pinosylvin).

A preparation according to the invention preferably contains a total content of hydroxystilbene-containing plant extracts of 0.1 to 5% by weight, in particular 0.1 to 4% by weight, preferably 0.5 to 3% by weight, particularly preferably 1 to 2% by weight.

Hydroxystilbene-containing plant extracts suitable according to the invention are selected from extracts from Vitaceae, Umbelliferae, Myrtaceae, Dipterocarpaceae, Cyperaceae, Gnetaceae, legumes, Gramineae, Sericeae, Haemodoraceae, Musaceae, Polygonaceae, Pinaceae, Cupressaceae, Cesalpiniaceae, Poaceae, Solanaceae, in particular Balanocarpus zeylanicus, Caragana chamlagu, Caragana sinica, Carex fedia, Carex humilis, Carex kobomugi, Carex pendula, Carex pumila Thunb, Cyphostemma crotalarioides, Gnetum hainanense, Gnetum ula, Gnetum venosum, Hopea parviflora, Iris clarkei, Neobalanocarpus heimii, Paeoni lactiflora, Parthenocissus tricuspidata, Polygonum cus pidatum, Polygonum multiflorum, Shorea disticha, Shorea hemsleyana, Sophora davidii, Sophora leachiana, Sophora nuttaliana, Vatica rassak, Vatica affinis, Vitis amurensis, Vitis betulifolia, Vitis flexuosa, Vitis heyneana, Vitis quinquangularis, Vitis coignetiae and Vitis vinifera, particularly trans-epsilon-viniferine (CAS: 62218-08-0) in free form or as a glycoside.

Hydroxystilbene-containing plant extracts which are particularly suitable according to the invention are extracts from needles of the Pinophyta (coniferous family), in particular from the needles of the pine family (Pinaceae), preferably the pine species Pinus ponderosa, Pinus albicaulis, Pinus aristata, Pinus attenuata, Pinus balfouriana, Pinus banksiana, Pinus bungeana, Pinus cembra, Pinus cembroides, Pinus clausa, Pinus contorta, Pinus coulteri, Pinus densiflora, Pinus echinata, Pinus edulis, Pinus elliottii, Pinus engelmannii, Pinus flexilis, Pinus glabra, Pinus heldreichii, Pinus jeffreyi, Pinus lambertiana, Pinus longaeva, Pinus massoniana, Pinus monophylla, Pinus monticola, Pinus mugo, Pinus muricata, Pinus nigra, Pinus palustris, Pinus parviflora, Pinus pungens, Pinus quadrifolia, Pinus radiata, Pinus resinosa, Pinus rigida, Pinus sabiniana, Pinus serotina, Pinus strobiformis, Pinus strobus, Pinus sylvestris, Pinus tabulaeformis, Pinus taeda, Pinus thun-bergiana, Pinus torreyana, Pinus virginiana, Pinus yuannensis and Pinus washoensis.

Particularly preferred according to the invention is an extract from Pinus cembra, with the extract available from Mibelle AG under the name Pinolumin being very particularly preferred.

A preparation according to the invention preferably contains a total glacier water content of 1 to 20% by weight, in particular 5 to 15% by weight, preferably 7 to 13% by weight, particularly preferably 10% by weight.

According to the invention, glacier water is understood to mean water that comes from glaciers. Glacier water froze at a time when environmental toxins were not yet polluting the waters. Due to the annual movement of the glacier over the rock, the water is enriched with valuable minerals, but is toxicologically very pure and free of harmful environmental influences.

Glacier waters preferred according to the invention are in particular alpine glacier waters, preferably selected from
Glacier water from Mont Blanc, available from Greentech GmbH, Starnberg under the name “Argentiere Ice Cap water”,
liposmomal encapsulated Swiss glacier water, available from Mibelle AG (Switzerland) under the name “Lipobelle Glacier”,
Glacier water from Switzerland, available from Botanica GmbH (Switzerland) and
Glacier water from the Monte Rosa massif in Italy, available from Symrise, Holzminden under the name “Extrapone® Glacier Water GW”.
Glacier water without the addition of preservatives listed in the currently valid European cosmetics regulations.

The preparation according to the invention advantageously brings about a cosmetic improvement in the appearance of the skin and an improvement in the condition of the skin. In particular, the preparation according to the invention has an anti-aging effect and improves the appearance of cellulite skin.

The preparation according to the invention can be made up in any form that seems suitable to the person skilled in the art, but it is particularly preferably made up as an ointment or gel.

A further subject of the present invention is the use of a preparation according to the invention in the field of pharmacy or medicine for dermatological skin care and to improve the condition of the skin.

A further subject of the present invention is a process for producing a preparation according to the invention for skin care and for improving the condition of the skin, characterized in that the ingredients mentioned in claim 1, as described for the preparations according to the invention, are mixed with cosmetically or pharmacologically suitable and tolerated carriers.

For the purposes of the present invention, the ingredients mentioned in claim 1 are preferably introduced or incorporated as components into a cosmetic or pharmaceutical preparation.

Depending on the type of formulation, the pharmaceutical preparation according to the invention can contain at least one further auxiliary or additive, such as, for example, oils, protective colloids, plasticizers, antioxidants and/or emulsifiers.

In the case of a dispersion, particularly in the case of a suspension or emulsion, it is advantageous to additionally use a physiologically tolerated oil such as sesame oil, corn oil, cottonseed oil, kahai oil, soybean oil or peanut oil, esters of medium-chain vegetable fatty acids or fish oils such as mackerel, sprat or salmon oil.

To increase the stability of the active ingredient against oxidative degradation, it is advantageous to add stabilizers such as α-tocopherol, β-tocopherol, γ-tocopherol, δ-tocopherol, gallates, diethylhexyl syringylidenemalonate, ascorbic acid and its esters, catechin, carnosic acid, genistein, lipoic acid, kaempferol, caffeic acid, oleuropein, hydroxytyrosol, quercetin, rosmarinic acid, sinapic acid, bis-ethylhexyl hydroxydimethoxy benzylmalonate, tetrasodium glutamate diacetate, sodium polyphosphate, epigallocatechin gallate, beta-carotene, ectoine or lycopene.

The dosage and duration of use of the preparation according to the invention can be appropriately adjusted and varied by the person skilled in the art.

The possible dosage forms of the preparation according to the invention include topical or local application forms in the form of plasters, rinses, shampoos, foam or shower baths, gels, ointments, creams, lotions, stick preparations, powders, pastes and sprays, as well as aerosol applications of the aforementioned application forms.

The cosmetic and pharmaceutical preparations according to the invention, such as hair shampoos, hair lotions, foam baths, shower baths, creams, gels, lotions, alcoholic and aqueous/alcoholic solutions, emulsions, wax/fat masses, stick preparations, powders or ointments can—depending on the type of formulation—as auxiliaries and additives contain mild surfactants, oil bodies, emulsifiers, superfatting agents, pearlescent waxes, consistency agents, thickeners, polymers, silicone compounds, fats, waxes, stabilizers, biogenic active ingredients, deodorants, antiperspirants, anti-dandruff agents, film formers, swelling agents, UV light protection factors, antioxidants, hydrotropes, preservatives, insect repellents, self-tanners, solubilizers, perfume oils, dyes and the like.

Typical examples of suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamido betaines and/or protein fatty acid condensates, the latter preferably based on wheat proteins or rice proteins.

Taken into consideration as oil bodies, for example, are Guerbet alcohols based on fatty alcohols with 6 to 18, preferably 8 to 10 carbon atoms, esters of linear C₆-C₃₂ fatty acids with linear C₆-C₃₂ fatty alcohols, esters of branched C₆-C₃₂ carboxylic acids with linear C₆-C₃₂ fatty alcohols, such as myristyl myristate, myristyl palmitate, myristyl stearate, myristyl isostearate, myristyl oleate, myristyl behenate, myristyl arucate, cetyl myristate, cetyl palmitate, cetyl stearate, cetyl isostearate, cetyl oleate, cetyl behenate, cetyl rucate, stearyl myristate, stearyl palmitate, stearyl stearate, stearyl isostearate, stearyl oleate, stearyl behenate, stearyl rucate, isostearyl myristate, isostearyl palmitate, isostearyl stearate, isostearyl isostearate, isostearyl oleate, isostearyl behenate, isostearyl oleate[Translator: repeated], oleyl myristate, oleyl palmitate, oleyl stearate, oleyl isostearate, oleyl oleate, oleyl behenate, oleyl rucate, behenyl myristate, behenyl palmitate, behenyl stearate, behenyl isostearate, behenyl oleate, behenyl behenate, erucyl myristate, erucyl palmitate, erucyl stearate, erucyl isostearate, erucyl oleate, erucyl behenate and erucyl erucate.

Also suitable are esters of linear C₆-C₃₂ fatty acids with branched alcohols, in particular 2-ethylhexanol, esters of hydroxycarboxylic acids with linear or branched C₆-C₃₂ fatty alcohols, in particular dioctyl malates, esters of linear and/or branched fatty acids with polyvalent alcohols (such as, for example, propylene glycol, dimerdiol or trimertriol) and/or Guerbet alcohols, triglycerides based on C₆-C₁₀ fatty acids, liquid mono-/di-/triglyceride mixtures based on C₆-C₁₈ fatty acids, esters of C₆-C₃₂ fatty alcohols and/or Guerbet alcohols with aromatic carboxylic acids, in particular benzoic acid, esters of C₂-C₁₂ dicarboxylic acids with linear or branched alcohols with 1 to 32 carbon atoms or polyols with 2 to 10 carbon atoms and 2 to 6 hydroxyl groups, vegetable oils, branched primary alcohols, substituted cyclohexanes, linear and branched C₆-C₃₂ fatty alcohol carbonates, Guerbet carbonates, esters of benzoic acid with linear and/or branched C₆-C₃₂ alcohols (e.g. Finsolv® TN), linear or branched, symmetrical or asymmetrical dialkyl ethers with 6 to 32 carbon atoms per alkyl group, ring opening products of epoxidized fatty acid esters with polyols, silicone oils and/or aliphatic or naphthenic hydrocarbons, such as, for example, squalane, squalene, hemisqualane or dialkylcyclohexane.

Suitable emulsifiers include, for example, non-ionic surfactants from at least one of the following groups:
(1) addition products of 2 to 30 moles of ethylene oxide and/or 0 to 5 moles of propylene oxide with linear fatty alcohols with 8 to 32 carbon atoms, with fatty acids with 12 to 32 carbon atoms, with alkylphenols with 8 to 15 carbon atoms in the alkyl group and alkylamines with 8 to 32 carbon atoms in the alkyl radical;
(2) C₁₂-C₁₈ fatty acid mono- and diesters of addition products of 1 to 30 mol of ethylene oxide with glycerol;
(3) glycerol mono- and diesters and sorbitan mono- and diesters of saturated and unsaturated fatty acids with 6 to 32 carbon atoms and their ethylene oxide addition products;
(4) alkyl and/or alkenyl mono- and oligoglycosides with 8 to 32 carbon atoms in the alk(en)yl radical and their ethoxylated analogs;
(5) addition products of 15 to 60 mol of ethylene oxide with castor oil and/or hydrogenated castor oil;
(6) polyol and especially polyglycerol esters;
(7) Partial esters based on linear, branched, unsaturated or saturated C/32 fatty acids, ricinoleic acid as well as 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
(8) mono-, di- and trialkyl phosphates as well as mono-, di- and/or tri-PEG alkyl phosphates and their salts:
(9) wool wax alcohols;
(10) polysiloxane-polyalkyl-polyether copolymers or corresponding derivatives;
(11) mixed esters of pentaerythritol, fatty acids, citric acid and fatty alcohol according to DE 1165574 PS and/or mixed esters of fatty acids with 6 to 32 carbon atoms, methyl glucose and polyols, preferably glycerol or polyglycerol,
(12) polyalkylene glycols as well as
(13) glycerol carbonate.

The addition products of ethylene oxide and/or propylene oxide with fatty alcohols, fatty acids, alkylphenols, glycerol mono- and diesters as well as sorbitan mono- and diesters of fatty acids or with castor oil represent known, commercially available products.

These are homologous mixtures whose average degree of alkoxylation corresponds to the ratio of the amounts of ethylene oxide and/or propylene oxide and substrate with which the addition reaction is carried out. C_{12/18} fatty acid mono- and diesters of addition products of ethylene oxide with glycerol are known from DE 2024051 PS as refatting agents for cosmetic preparations.

Alkyl and/or alkenyl mono- and oligoglycosides, their production and their use are known from the prior art. They are produced in particular by reacting glucose or oligosaccharides with primary alcohols with 8 to 18 carbon atoms. With regard to the glycoside residue, both monoglycosides, in which a cyclic sugar residue is glycosidically bonded to the fatty alcohol, as well as oligomeric glycosides with a degree of oligomerization up to preferably about 8 are suitable. The degree of oligomerization is a statistical average, which is based on a homolog distribution that is usual for such technical products.

Typical examples of suitable polyglycerol esters are polyglyceryl-2 dipolyhydroxystearates (Dehymuls® PGPH), polyglyceryl-3-diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Beilina®), polyglyceryl-4 caprate (polyglycerol caprate T2010/90), polyglyceryl-3 cetyl ether (Chimexane® NL), polyglyceryl-3 distearates (Cremophor® GS 32) and polyglyceryl polyricinoleate (Admul® WOL 1403), polyglyceryl dimerate isostearate and mixtures thereof.

Zwitterionic surfactants can also be used as emulsifiers. Zwitterionic surfactants are those surface-active compounds that have at least one quaternary ammonium group and at least one carboxylate and one sulfonate group in the molecule. Particularly suitable zwitterionic surfactants are the so-called betaines such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines each with 8 to 18 carbon atoms in the alkyl or acyl group as well as the cocoacylaminoethyl hydroxyethyl carboxymethyl glycinate. The fatty acid amide derivative known under the CTFA name Cocamidopropyl Betaine is particularly preferred. Ampholytic surfactants are also suitable emulsifiers. Ampholytic surfactants are those surface-active compounds which, in addition to a C_{8/18} alkyl or acyl group, contain at least one free amino group and at least one COOH or SO₃H group in the molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids, each with about 8 to 18 C atoms in the alkyl group. Particularly preferred ampholytic surfactants are N-cocoalkylaminopropionate, cocoacylaminoethylaminopropionate and C_{12/18}-acylsarcosine. In addition to the ampholytic ones, quaternary emulsifiers can also be considered, with those of the esterquat type, preferably methyl-quaternized difatty acid triethanolamine ester salts, being particularly preferred.

Substances such as lanolin and lecithin as well as polyethoxylated or acylated lanolin and lecithin derivatives, polyol fatty acid esters, monoglycerides and fatty acid alkanolamides can be used as superfatting agents, the latter also serving as foam stabilizers. Lanolin substitutes can also be used as moisturizers. The materials Pelemol VL and Pelemol CA from Phoenix, Kahlwax 6421 from Kahl and Vegenolin CF from MMP are particularly suitable here.

Possible pearlescent waxes include: alkylene glycol esters, especially ethylene glycol distearate; fatty acid alkanolamides, especially coconut fatty acid diethanolamide; partial glycerides, especially stearic acid monoglyceride; esters of polyvalent, optionally hydroxy-substituted carboxylic acids with fatty alcohols with 6 to 32 carbon atoms, especially long-chain esters of tartaric acid; fatty substances, such as fatty alcohols, fatty ketones, fatty aldehydes, fatty ethers and fatty carbonates, which have a total of at least 24 carbon atoms, especially lauron and distearyl ether; fatty acids such as stearic acid, hydroxystearic acid or behenic acid, ring opening products of olefin epoxides with 12 to 32 carbon atoms with fatty alcohols with 12 to 32 carbon atoms and/or polyols with 2 to 15 carbon atoms and 2 to 10 hydroxyl groups and mixtures thereof.

Primarily fatty alcohols or hydroxy fatty alcohols with 12 to 32 and preferably 16 to 18 carbon atoms and also partial glycerides, fatty acids or hydroxy fatty acids come into consideration as consistency agents. A combination of these substances with alkyl oligoglucosides and/or fatty acid N-methylglucamides of the same chain length and/or polyglycerol poly-12-hydroxystearates is preferred. Substances which, in a concentration of 1-10%, preferably 2-5%, increase the viscosity of the preparation by at least 5000 mPas, preferably by 2000-4000 mPas (Brookfeld viscometer, type DV3-T) are also accepted as consistency agents. The measuring conditions depend on the respective forms of application:

| | Spray, | | | | Lip balm, |
|---|---|---|---|---|---|
| | Oils | Shampoo | Lotion | Cream | Butter |
| Vessel | 100 ml | 100 ml | 100 ml | 30 ml | 30 ml |
| | screw- | jar | jar | screw- | screw- |
| | top jar | | | top jar | top jar |
| Spindle no. | RV-02 (2) | RV-04 (4) | RV-06 (6) | T-E (95) | T-E (95) |
| Rotation | 20 | 20 | 10 | 5 | 5 |
| [rpm] | | | | | |
| Measuring | 30 | 30 | 30 | 30 | 30 |
| time [sec] | | | | | |

Suitable thickeners are, for example, Aerosil types (hydrophilic silicas), polysaccharides, in particular xanthan gum, guar-guar, agar-agar, alginates and tyloses, pectins, sclerotium gum, Acacia Senegal gum, Caesalpinia spinosa gum, carboxymethyl cellulose and hydroxyethyl cellulose, also higher molecular weight polyethylene glycol mono- and diesters of fatty acids, polyacrylates (e.g. Carbopole® from Lubrizol or Synthalene® from Sigma), polyacrylamides, polyvinyl alcohol and polyvinylpyrrolidone, surfactants such as ethoxylated fatty acid glycerides, esters of fatty acids with polyols such as pentaerythritol or trimethylolpropane, fatty alcohol ethoxylates with a narrow homolog distribution or alkyl oligoglucosides as well as electrolytes such as table salt and ammonium chloride.

Suitable cationic polymers are, for example, cationic cellulose derivatives, such as a quaternized hydroxyethyl cellulose, which is available under the name Polymer JR 400® from Amerchol, cationic starch, copolymers of diallylammonium salts and acrylamides, quaternized vinylpyrrolidone/vinylimidazole polymers, such as Luviquat® (BASF), condensation products of polyglycols and amines, quaternized collagen polypeptides, such as lauryldimonium hydroxypropyl hydrolyzed collagen (Lamequat® L/Grunau), quaternized wheat polypeptides, polyethyleneimine, cationic silicone polymers, such as amidomethicone, copolymers of adipic acid and dimethylaminohydroxypropyldiethylenetriamine (Cartaretine®/Sandoz), copolymers of acrylic acid with dimethyl diallylammonium chloride (Merquat® 550/Chemviron), polyaminopolyamides, such as described in FR 32 2252840 A and their cross-linked water-soluble polymers, cationic chitin derivates such as quaternized chitosan, optionally distributed in microcrystalline form, condensation products of dihaloalkyls, such as dibromobutane with bisdialkylamines, such as bis-dimethylamino-1,3-propane, cationic guar gum, such as Jaguar® CBS, Jaguar® C-17, Jaguar® C-16 from Celanese, quaternized ammonium salt polymers, such as Mirapol® A-15, Mirapol® AD-1, Mirapol® AZ-1 from Miranol.

Anionic, zwitterionic, amphoteric and nonionic polymers include, for example, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and their esters, non-cross-linked polyacrylic acids and polyacrylic acids cross-linked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinylcaprolactam terpolymers and optionally derivatized cellulose ethers and silicones.

Suitable silicone compounds are, for example, dimethylpolysiloxanes, methylphenylpolysiloxanes, cyclic silicones and amino-, fatty acid-, alcohol-, polyether-, epoxy-, fluorine-, glycoside- and/or alkyl-modified silicone compounds, which can be in both liquid and resinous form at room temperature. Also suitable are simethicones, which are mixtures of dimethicones with an average chain length of 200 to 300 dimethylsiloxane units and hydrogenated silicates. A detailed overview of suitable volatile silicones can also be found by Todd et al. in Cosm. Toil. 91, 27 (1976).

Typical examples of fats are glycerides, and waxes include natural waxes, such as candelilla wax, carnauba wax, Japanese wax, esparto grass wax, cork wax, guaruma wax, rice germ oil wax, sugar cane wax, ouricury wax, montan wax, beeswax, shellac wax, spermaceti, lanolin (wool wax), rump fat, ceresin, ozokerite (earth wax), petrolatum, hydrogenated castor oil, hydrogenated vegetable oils (e.g. Dermofeel Viscolid from Evonik), paraffin waxes, microwaxes; chemically modified waxes (hard waxes), such as Montanester waxes, Sasol waxes, Fischer-Tropsch waxes, hydrogenated jojoba waxes and synthetic waxes, such as polyalkylene waxes and polyethylene glycol waxes.

Metal salts of fatty acids, such as magnesium, aluminum and/or zinc stearate or ricinoleate, can be used as stabilizers.

Biogenic active ingredients include, for example, tocopherol, tocopherol acetate, tocopherol palmitate, ascorbic acid, deoxyribonucleic acid, retinol, bisabolol, allantoin, phytantriol, panthenol, AHA acids, amino acids, hydroxytyrosol, carotene, sinapic acid, rapeseed oil, lycopene, ceramides, pseudoceramides, essential oils, plant extracts and vitamin complexes.

Cosmetic deodorants (deodorants) counteract, mask or eliminate body odors. Body odors arise from the action of skin bacteria on apocrine sweat, whereby unpleasant-smelling degradation products are formed. Accordingly, deodorants contain active ingredients that act as germ-inhibiting agents, enzyme inhibitors, odor absorbers or odor maskers.

In principle, all substances that are effective against gram-positive bacteria are suitable as germ-inhibiting agents, which may be added to the cosmetics according to the invention, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (Triclosan), 4-chloro-3,5-dimethylphenol, 2,2′-methylene-bis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynylbutylcarbamate, chlorhexidine, 3,4,4′-trichlorocarbanilide (TTC), antibacterial fragrances, thymol, thyme oil, eugenol, clove oil, menthol, mint oil, farnesol, phenoxyethanol, glycerol monolaurate (GML), diglycerol monocaprinate (DMC), salicylic acid N-alkylamides such as salicylic acid n-octylamide or salicylic acid n-decylamide.

Enzyme inhibitors can also be added to the cosmetics according to the invention. Suitable enzyme inhibitors are, for example, esterase inhibitors. These are preferably trialkyl citrates such as trimethyl citrate, tripropyl citrate, triisopropyl citrate, tributyl citrate and in particular triethyl citrate (Hydagen® CAT, BASF AG). The substances inhibit enzyme activity and thereby reduce odor formation. Other substances that can be used as esterase inhibitors are sterol sulfates or phosphates, such as lanosterol, cholesterol, campesterol, stigmasterol and sitosterol sulfate or phosphate, dicarboxylic acids and their esters, such as glutaric acid, glutaric acid monoethyl ester, glutaric acid diethyl ester, adipic acid, monoethyl adipate, diethyl adipate, malonic acid and diethyl malonate, hydroxycarboxylic acids and their esters such as citric acid, malic acid, tartaric acid or diethyl tartaric acid, and zinc glycinate.

Substances that can absorb odor-causing compounds and largely retain them are suitable as odor absorbers. They lower the partial pressure of the individual components and thus also reduce their propagation speed. It is important that perfumes remain unaffected. Odor absorbers are not effective against bacteria. For example, they contain as their main component a complex zinc salt of ricinoleic acid or special, largely odorless fragrances that are known to the person skilled in the art as “fixators”, such as extracts of labdanum or styrax or certain abietic acid derivatives. Fragrances or perfume oils act as odor maskers and, in addition to their function as odor maskers, give the deodorants their respective scent. Examples of perfume oils that may be mentioned are mixtures of natural and synthetic fragrances. Natural fragrances are extracts from flowers, stems and leaves, fruits, fruit peels, roots, woods, herbs and grasses, needles and twigs as well as resins and balms. Animal raw materials also come into consideration, such as civet and castoreum. Typical synthetic fragrance compounds are products of the ester, ether, aldehyde, ketone, alcohol and hydrocarbon types. Fragrance compounds of the ester type are, for example, benzyl acetate, p-tert-butylcyclohexyl acetate, linalyl acetate, phenylethyl acetate, linalyl benzoate, benzyl formate, allylcyclohexyl propionate, styrallyl propionate and benzyl salicylate. The ethers include, for example, benzyl ethyl ether, the aldehydes include, for example, the linear alkanals with 8 to 18 carbon atoms, citral, citronellal, citronellyloxyacetaldehyde, cyclamenaldehyde, hydroxycitronellal, lilial and bourgeonal, the ketones include, for example, the ionones and methylcedryl ketone, the alcohols include anethole, citronellol, eugenol, isoeugenol, geraniol, linalool, phenylethyl alcohol and terpineol, the hydrocarbons mainly include terpenes and balms. However, mixtures of different fragrances are preferably used, which together produce an appealing scent. Essential oils of lower volatility, which are mostly used as aroma components, are also suitable as perfume oils, e.g. sage oil, chamomile oil, clove oil, lemon balm oil, mint oil, cinnamon leaf oil, lime blossom oil, juniper berry oil, vetiver oil, oliban oil, galbanum oil,
labdanum oil and lavandin oil. Bergamot oil, dihydromyrcenol, lilial, lyral, citronellol, phenylethyl alcohol, α-hexyl cinnamaldehyde, geraniol, benzylacetone, cyclamenaldehyde, linalool, boisambrene forte, ambroxan, indole, hedione, sandelice, lemon oil, tangerine oil, orange oil, muguet alcohol, majantol, mugetanol, allylamyl glycolate, cyclovertal, lavandin oil, clary sage oil, ß-damascone, geranium oil bourbon, cyclohexyl salicylate, Vertofix Coeur, Iso-E-Super, Fixolide NP, Evernyl, Iraldein gamma, phenylacetic acid, geranyl acetate, benzyl acetate, rose oxide, Romilat, Irotyl and Floramat are preferably used alone or in mixtures.

Antiperspirants reduce sweat production by influencing the activity of the eccrine sweat glands, thus counteracting armpit wetness and body odor. Aqueous or water-free formulations of antiperspirants typically contain the following ingredients:
(a) astringent active ingredients,
(b) oil components,
(c) nonionic emulsifiers,
(d) coemulsifiers,
(e) consistency agents,
(f) auxiliary materials such as thickeners or complexing agents and/or
(g) non-aqueous solvents such as ethanol, butylene glycol, pentylene glycol, propanediol, methylpropanediol, hexanediol, propylene glycol and/or glycerin.

Salts of aluminum, zirconium or zinc are particularly suitable as astringent antiperspirant active ingredients. Such suitable antihydrotically active ingredients are, for example, aluminum chloride, aluminum chlorohydrate, aluminum dichlorohydrate, aluminum sesquichlorohydrate and their complex compounds, for example, with propylene glycol-1,2. Aluminum hydroxyallantoinate, aluminum chloride tartrate, aluminum zirconium trichlorohydrate, aluminum zirconium tetrachlorohydrate, aluminum zirconium pentachlorohydrate and their complex compounds e.g. with amino acids such as glycine.

In addition, common oil-soluble and water-soluble additives can be contained in smaller amounts in antiperspirants. Such oil-soluble auxiliary materials can be, for example:
anti-inflammatory, skin-protecting or fragrant essential oils,
synthetic skin-protecting active ingredients and/or
oil-soluble perfume oils.

Common water-soluble additives are, for example, preservatives, water-soluble fragrances, pH value adjusters, for example buffer mixtures, water-soluble thickeners, for example water-soluble natural or synthetic polymers such as xanthan gum, hydroxyethyl cellulose, pectins, cross-linked hyaluronic acid, acetylated hyaluronic acid, hyaluronic acids with different molecular weights of 10-1.8 million Dalton, carrageenan, polyvinyl alcohols, alginates, cellulose, hyaluronic acid, polyvinylpyrrolidone or high molecular weight polyethylene oxides.

Climbazole, Octopirox, salicylic acid, willow bark extract and zinc pyrithione can be used as anti-dandruff agents.

Common film formers include chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid or its salts and similar compounds.

Montmorillonite, clay minerals, pemulene and alkyl-modified Carbopol types (Lubrizol) can serve as swelling agents for aqueous phases. Other suitable polymers or swelling agents can be found in the overview by R. Lochhead in Cosm. Toil.
108, 95 (1993).

UV light protection factors are, for example, organic substances (light protection filters) that are liquid or crystalline at room temperature and are able to absorb ultraviolet rays and release the absorbed energy in the form of longer-wave radiation, e.g. heat. UVB filters can be oil-soluble or water-soluble. Examples of oil-soluble substances include:
3-benzylidene camphor or 3-benzylidene norcamphor and its derivatives, for example 3-(4-methylbenzylidene) camphor as described in EP 0693471 E1;
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester;
Esters of cinnamic acid, preferably 4-methoxycinnamic acid 2-ethylhexyl ester, 4-methoxycinnamic acid propyl ester, 4-methoxycinnamic acid isoamyl ester, 2-cyano-phenylcinnamic acid-2-ethylhexylester (Octocrylene);
Esters of salicylic acid, preferably salicylic acid 2-ethylhexyl ester, salicylic acid 4-isopropyl benzyl ester, salicylic acid homomenthyl ester;
Derivatives of benzophenone, preferably 2-hydroxy-4-methoxybenzophenone, 2-hydroxy-4-methoxy-4′-methylbenzophenone, 2,2′-dihydroxy-4-methoxybenzophenone;
Esters of benzalmalonic acid, preferably 4-methoxybenzmalonic acid di-2-ethylhexyl ester;
Triazine derivatives, such as 2,4,6-trianilino-(p-carbo-2′-ethyl-1′-hexyloxy)-1,3,5-triazine and octyl triazone, as described in EP 0818450 A1 or dioctyl butamido triazone (Uvasorb® HEB);
propane-1,3-diones, such as 1-(4-tert-butylphenyl)-3-4′-methoxyphenyl)propanedione;
Ketotricyclo(5.2.1.0)decane derivatives, as described in EP 0694521 E1.

Possible water-soluble substances are:
2-phenylbenzimidazole-5-sulfonic acid and its alkali, alkaline earth, ammonium, alkylammonium, alkanolammonium and glucammonium salts;
sulfonic acid derivatives of benzophenones, preferably 2-hydroxy-4-methoxybenzophenone-5-sulfonic acid and its salts;
Sulfonic acid derivatives of 3-benzylidene camphor, such as 4-(2-oxo-3-bornylidenemethyl)benzenesulfonic acid and 2-methyl-5-(2-oxo-3-bornylidene)sulfonic acid and their salts.

Particularly suitable as typical UV-A filters are derivatives of benzoylmethane, such as 1-(4′-tert-butylphenyl)-3-(4′-methoxyphenyl)propane-1,3-dione, 4-tert-butyl-4′-methoxydibenzoylmethane (Parsol 1789), 1-phenyl-3-(4′-isopropylphenyl)-propane-1,3-dione and enamine compounds, as described in DE 19712033 A1 (BASF). The IIV-A and UV-B filters can of course also be used in mixtures. In addition to the soluble substances mentioned, insoluble light protection pigments, namely finely dispersed metal oxides or salts, can also be used for this purpose. Examples of suitable metal oxides are, in particular, zinc oxide and titanium dioxide and also oxides of iron, zirconium, silicon, manganese, aluminum and cerium and mixtures thereof. Silicates (talc), barium sulfate or zinc stearate can be used as salts. The oxides and salts are used in the form of pigments for skin care and skin-protecting emulsions and decorative cosmetics. The particles should have an average diameter of less than 100 nm, preferably between 5 and 50 nm and in particular between 15 and 30 nm. They can have a spherical shape, but particles can also be used that have an ellipsoidal shape or a shape that deviates from the spherical shape in some other way. The pigments can also be surface-treated, i.e. hydrophilized or hydrophobic. Typical examples are coated titanium dioxides, such as titanium dioxide T 805 (Evonik) or Eusolex® T2000 (Merck). Silicones and especially trialkoxyoctylsilanes or simethicones are particularly suitable as hydrophobic coating agents. So-called micro- or nano-pigments are preferably used in sunscreens. Ironized zinc oxide is preferably used. Further suitable UV light protection filters can be found in the overview by P. Finkel in SÖFW-Journal 122, 543 (1996).

IR absorbers or HEV light filters can also be considered light protection substances (e.g. lutein available as FloraGlo from Kemin or Dragosin from Symrise).

In addition to the two groups of primary sunscreens mentioned above, secondary sunscreens of the antioxidant type can also be used, which interrupt the photochemical reaction chain that is triggered when UV radiation penetrates the skin. Typical examples of this are amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearylthiodipropionate, thiodipropionic acid and its derivatives (esters, ethers, peptides, lipids, nucleotides, nucleosides and salts) as well as sulfoximine compounds (e.g. buthionine sulfoximine, homocysteine sulfoximine, butionine sulfones, penta-, hexa-, heptathionine sulfoximine) in very low tolerable doses (e.g. pmol to pmol/kg), as well as (metal) chelators (e.g. a-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and its derivatives, vitamin C and derivatives (e.g. ascorbyl palmitate, Mg-ascorbyl phosphate, ascorbyl acetate, sodium ascorbyl phosphate, ascorbyl isostearate), tocopherols and derivatives (e.g. vitamin E acetate), vitamin A and derivatives (vitamin A palmitate) as well as coniferyl benzoate of benzoin resin, rutinic acid and its derivatives, α-glycosylrutin, ferulic acid, furfurylidene glucitol, carnosine, butylhydroxytoluene, butylhydroxyanisole, nordihydroguaiac acid, nordihydroguajaretic acid, trihydroxybutyrophenone, uric acid and its derivatives, mannose and its derivatives, superoxide dismutase, zinc and its derivatives (e.g. ZnO, ZnSO₄), selenium and its derivatives (e.g. Selenium-methionine), stilbenes and their derivatives (e.g. stilbene oxide, trans-stilbene oxide) and the derivatives suitable according to the invention (salts, esters, ethers, sugars, nucleotides, nucleosides, peptides and lipids) of these active ingredients mentioned.

To improve the flow behavior, hydrotropes such as ethanol, isopropyl alcohol, or polyols can also be used. Polyols that come into consideration here preferably have 2 to 15 carbon atoms and at least two hydroxyl groups. The polyols can contain further functional groups, in particular amino groups, or can be modified with nitrogen. Typical examples are
glycerin;
alkylene glycols, such as ethylene glycol, diethylene glycol, propylene glycol, butylene glycol, pentylene glycol, hexylene glycol and polyethylene glycols with an average molecular weight of 100 to 1,000 daltons;
technical oligoglycerol mixtures with a degree of inherent condensation of 1.5 to 10, such as technical diglycerol mixtures with a diglycerol content of 40 to 50% by weight;
methyl compounds, such as in particular trimethylol ethane, trimethylol propane, trimethylol butane, pentaerythritol and dipentaerythritol;
lower alkyl glucosides, especially those with 1 to 8 carbons in the alkyl radical, such as methyl and butyl glucoside;
sugar alcohols with 5 to 12 carbon atoms, such as sorbitol or mannitol,
sugars with 5 to 12 carbon atoms, such as glucose or sucrose;
amino sugars such as glucamine;
dialcohol amines, such as diethanolamine or 2-amino-1,3-propanediol.
magnolia extract at least 98%
perilla acid
hydroxyacetophenones

Examples of suitable preservatives are phenoxyethanol, formaldehyde solution, parabens, pentanediol or sorbic acid, as well as the other substance classes listed in Appendix 6, Parts A and B of the Cosmetics Ordinance. N,N-diethyl-m-toluamide, 1,2-pentanediol or ethyl butylacetylaminopropionate can be used as insect repellents; dihydroxyacetone or erythrulose are suitable as self-tanners.

Perfume oils that may be mentioned are mixtures of natural and synthetic fragrances. Natural fragrances include extracts of flowers (lily, lavender, roses, jasmine, neroli, ylang-ylang), stems and leaves (geranium, patchouli, petitgrain), fruits (anise, coriander, caraway, juniper), fruit peels (bergamot, lemon, oranges), roots (mace, angelica, celery, cardamom, costus, iris, calmus), woods (pine, sandalwood, guaiac, cedar, rosewood), herbs and grasses (tarragon, lemongrass, sage, thyme), needles and twigs (spruce, fir, pine, mountain pine), resins and balms (galbanum, elemi, benzoin, myrrh, olibanum, opoponax). Animal raw materials also come into consideration, such as civet and castoreum. Typical synthetic fragrance compounds are products of the ester, ether, aldehyde, ketone, alcohol and hydrocarbon types. Fragrance compounds of the ester type are, for example, benzyl acetate, phenoxyethyl isobutyrate, p-tert-butylcyclohexyl acetate, linalyl acetate, dimethyl benzyl carbinyl acetate, phenyl ethyl acetate, linalyl benzoate, benzyl formate, ethyl methyl phenyl glycinate, allyl cyclohexyl propionate, styrallyl propionate and benzyl salicylate. The ethers include, for example, benzyl ethyl ether, the aldehydes include, for example, the linear alkanals with 8 to 18 carbon atoms, citral, citronellal, citronellyloxyacetaldehyde, cyclamenaldehyde, hydroxycitronellal, lilial and bourgeonal, the ketones include, for example, the ionones, α-isomethyl ionone and methylcedrylketone, the alcohols include anethole, citronellol, eugenol, isoeugenol, geraniol, linalool, phenylethyl alcohol and terpineol, the hydrocarbons mainly include the terpenes and balms. However, mixtures of different fragrances are preferably used, which together produce an appealing scent. Essential oils of lower volatility, which are mostly used as aroma components, are also suitable as perfume oils, e.g. sage oil, chamomile oil, clove oil, lemon balm oil, mint oil, cinnamon leaf oil, lime blossom oil, juniper berry oil, vetiver oil, olibanum oil, galbanum oil, labolanum oil and lavandin oil. Bergamot oil, dihydromyrcenol, lilial, lyral, citronellol, phenylethyl alcohol, α-hexyl cinnamaldehyde, geraniol, benzylacetone, cyclamenaldehyde, linalool, boisambrene forte, ambroxan, indole, hedione, sandelice, lemon oil, tangerine oil, orange oil, allylamyl glycolate, cyclovertal, lavandin oil, clary sage oil, ß-damascone, geranium oil bourbon, cyclohexyl salicylate, Vertofix Coeur, Iso-E-Super, Fixolide NP, Evernyl, Iraldein gamma, phenylacetic acid, geranyl acetate, benzyl acetate, rose oxide, Romilat, Irotyl and Floramat are preferably used alone or in mixtures.

Substances suitable and approved for cosmetic purposes can be used as dyes, such as those compiled, for example, in the publication “Cosmetic Dyes” of the Dye Commission of the German Research Foundation, Verlag Chemie, Weinheim, 1984, pp. 81-106. These dyes are usually used in concentrations of 0.001 to 1.0% by weight, based on the entire mixture.

The total proportion of auxiliary materials and additives can be 1 to 90, preferably 5 to 70% by weight, particularly preferably 10-35% by weight—based on the preparation. The cosmetic or pharmaceutical preparations can be produced using conventional cold or hot processes with or without high-pressure homogenization; the phase inversion temperature method is preferably used.

### EXAMPLES

The following recipe examples describe the invention without, however, restricting it thereto. Unless otherwise stated, all quantities, proportions and percentages are based on the weight and the total amount or the total weight of the preparation.

### Example 1: Eye Care Cream

| | | | Amount |
|---|---|---|---|
| Phase | Raw material | Inci | as % |
| Water | | | |
| phase | | | |
| | Water, demin | Aqua | 47.15 |
| | Glycerin 99.5% Vegan | Glycerin | 5.05 |
| | Zinc sulfate | Zinc sulfates | 1 |
| | heptahydrate | | |
| | Prodew 500 | Sodium PCA + Sodium Lactate + | 1 |
| | | Arginine + Aspartic Acid + PCA + Glycine + | |
| | | Alanine + Serine + Valine + Proline + | |
| | | Threonine + Isoleucine + Histidine + | |
| | | Phenylalanine + Aqua | |
| | Raya Hyaluron <10 | Sodium Hyaluronate | 0.5 |
| | Dalton | | |
| | Pinolumin pwd | Pinus Cembra Wood Extract + | 1 |
| | | Maltodextrin + Aqua | |
| | Majestem | Glycerin + Leontopodium Alpinum Callus | 2 |
| | | Culture Extract + Xanthan Gum | |
| | Symsave H | Hydroxyacetophenones | 0.5 |
| | Symsitive 1609 | Pentylene Glycol + 4-t-Butylcyclohexanol | 1 |
| | Snap-8 peptide | Aqua + Acetyl Octapeptide-3 + Caprylyl | 2 |
| | Solution C | Glycol | |
| | Glacier water | Aqua, Cinnamomum Cassia Bark Extract | 10 |
| | from Switzerland | | |
| Fat phase | | | |
| | Dermofeel PGPR | Polyglyceryl-3 Polyricinoleate | 3 |
| | Kahlwax 2811 | Oryza Sativa (Rice) Bran Wax | 3 |
| | Dermofeel MT 70 non | Tocopherol + Helianthus Annuus | 1 |
| | GMO | (Sunflower) Seed Oil | |
| | Cutina HR Powder | Hydrogenated Castor Oil | 1 |
| | Tegosoft DEC | Diethylhexyl Carbonate | 7 |
| | Cetiol Sensoft | Propylheptyl Caprylate | 5 |
| | Cetiol RLF | Caprylyl Caprylate/Caprate | 7 |
| | Cosphaderm Magnolia | Magnolia Officinalis Bark Extract | 1 |
| | Extract 98 | | |
| Perfume | | | |
| phase | | | |
| | Perfume Duigou Skin | Perfume | 0.8 |
| | Uni 26 | | |

### Example 2: Facial Serum

| | | | Amount |
|---|---|---|---|
| Phase | Raw material | Inci | as % |
| Water phase | | | |
| | Water, demin | Aqua | 73.3 |
| | Glacier water from | Aqua, | 10 |
| | Switzerland | Cinnamomum | |
| | | Cassia Bark | |
| | | Extract | |
| | Pinolumin pwd | Pinus Cembra | 3 |
| | | Wood Extract + | |
| | | Maltodextrin + | |
| | | Aqua | |
| | Snap-8 peptide | Aqua + Acetyl | 2 |
| | solution C | Octapeptide-3 + | |
| | | Caprylyl | |
| | | Glycol | |
| | Prodew 500 | Sodium PCA + | 0.5 |
| | | Sodium Lactate + | |
| | | Arginine + | |
| | | Aspartic | |
| | | Acid + PCA + | |
| | | Glycine + | |
| | | Alanine + Serine + | |
| | | Valine + Proline + | |
| | | Threonine + | |
| | | Isoleucine + | |
| | | Histidine + | |
| | | Phenylalanine + | |
| | | Aqua | |
| | Pullulan | Pullulan | 1 |

### Example 3: Facial Serum with Platinum Peptide

| | | | |
|---|---|---|---|
| | Resistem | Glycerin (and) | 2 |
| | | Globularia Cordifolia | |
| | | Callus Culture | |
| | | Extract | |
| | Et-VC | 3-0-Ethyl | 1 |
| | | Ascorbic Acid | |
| Thickener | | | |
| phase | | | |
| | Hydrolite 5 | Pentylene | 5 |
| | | Glycol | |
| | Raya Hyaluron <10 Dalton | Sodium | 0.9 |
| | | Hyaluronate | |
| | Raya Hyaluron LMW | Sodium | 0.8 |
| | | Hyaluronate | |
| Perfume | | | |
| phase | | | |
| | Perfume Duigou Skin Uni 26 | Perfume | 0.5 |

| | | | Amount |
|---|---|---|---|
| Phase | Raw material | Inci | as % |
| Water phase | | | |
| | Water, demin | Aqua | 70 |
| | Symsitive 1609 | Pentylene Glycol + 4-t- | 1 |
| | | butylcyclohexanol | |
| | Pinolumin pwd | Pinus Cembra Wood | 2 |
| | | Extract + Maltodextrin + | |
| | | Aqua | |
| | Argirelox peptide solution | Aqua + Glycerin + Acetyl | 2 |
| | | Hexapeptide- 8 + | |
| | | Pentapeptide-18 + Citric | |
| | | Acid + Caprylyl Glycol | |
| | Glacier water from Switzerland | Aqua, Cinnamomum | 10 |
| | | Cassia Bark Extract | |
| | MVP Platinum MatrixEM | Maltodextrin + Acetyl | 0.5 |
| | Powder | Tetrapeptide-17 + | |
| | | Collodial Platinum | |
| | Dermotenseur 2 | Propanediol + Aqua + | 2 |
| | | Kigelia Africana Fruit | |
| | | Extract + Quillaja | |
| | | Saponaria Bark Extract | |
| Thickener phase | | | |
| | Hydrolite 5 | Pentylene Glycol | 5 |
| | Raya Hyaluron <10 Dalton | Sodium Hyaluronate | 0.6 |
| | Sodium Hyaluronate HA-T | Sodium Hyaluronate | 0.9 |
| Perfume phase | | | |
| | Perfume Duigou Skin Uni 26 | Perfume | 0.5 |
| | Timiron Super Silver Fine | Mica + Titanium Dioxide | 0.5 |
| | Phytexcell Centella Asiatica | Glycerin + Butylene | 5 |
| | | Glycol + Aqua + Centella | |
| | | Asiatica Extract | |

### Example 4: Night Care Cream

| | | | Amount as |
|---|---|---|---|
| Phase | Raw material | Inci | % |
| Fat phase | | | |
| | Cold-pressed olive oil, | Olea Europaea (Olive) Fruit Oil | 2 |
| | organic | | |
| | Deodorized cocoa | Theobroma Cacao (Cocoa) Seed Butter | 2 |
| | butter, organic | | |
| | Sisterna SP 70-C | Sucrose Ester | 1 |
| | Cosphaderm | Magnolia Officinalis Bark Extract | 1 |
| | Magnolia Extract 98 | | |
| | Cetiol SB 45 | Butyrospermum Parkii (Shea) Butter | 1 |
| | Lanette 18 | Stearyl Alcohol | 3 |
| | Dermofeel GSC | Glyceryl Stearate Citrate | 5 |
| | Kahai Oil | Caryodendron Orinocense Seed Oil | 3 |
| | Mango butter, | Mangifera Indica (Mango) Seed Butter | 1 |
| | organic, deodorized | | |
| | Meadowfoam oil | Limnanthes Alba (Meadowfoam) Seed Oil | 3 |
| | DS-SBS | Glycerin + Stearic Acid + Macadamia | 2 |
| | | Ternifolia Seed Oil + Squalane + Stearyl | |
| | | Alcohol + Hydrogenated Lecithin + | |
| | | Ceramide 3 + Cholesterol + Cetyl Alcohol + | |
| | | Aqua | |
| | Juvinity | Caprylic/Capric Triglyceride + | 2 |
| | | Geranylgeranylisopropanol | |
| | Symsave H | Hydroxyacetophenones | 0.5 |
| Water | | | |
| phase | | | |
| | Water, demin | Aqua | 49.95 |
| | Glycerin 99.5% Vegan | Glycerin | 2 |
| | Glacier water from | Aqua, Cinnamomum Cassia Bark Extract | 10 |
| | Switzerland | | |
| | Pinolumin pwd | Pinus Cembra Wood Extract + Maltodextrin + | 2 |
| | | Aqua | |
| | Symsitive 1609 | Pentylene Glycol + 4-t-Butylcyclohexanol | 1 |
| Thickener | | | |
| phase | | | |
| | Raya Hyaluron LMW | Sodium Hyaluronate | 0.6 |
| | Argirelox peptide | Aqua + Glycerin + Acetyl Hexapeptide-8 + | 2 |
| | solution | Pentapeptide-18 + Citric Acid + Caprylyl | |
| | | Glycol | |
| | Raya Hyaluron <10 | Sodium Hyaluronate | 0.4 |
| | Dalton | | |
| | Zemea | Propanediol | 5 |
| Perfume | | | |
| phase | | | |
| | Perfum Duigou | Perfume | 0.55 |
| | Skin Uni 26 | | |

### Example 5: Day Care Cream

| | | | Amount as |
|---|---|---|---|
| Phase | Raw material | Inci | % |
| Fat phase | | | |
| | Avocado oil, organic | Persea Gratissima | 1 |
| | | (Avocado) Oil | |
| | Cocoa butter, deodorized, organic | Theobroma Cacao (Cocoa) | 1 |
| | | Seed Butter | |
| | Symsave H | Hydroxyacetophenones | 0.5 |
| | Sisterna SP 70-C | Sucrose Ester | 1 |
| | Lanette 0 | Cetearyl Alcohol | 3 |
| | Kahai Oil | Caryodendron Orinocense | 2 |
| | | Seed Oil | |
| | Dermofeel GSC | Glyceryl Stearate Citrate | 5 |
| | Cosphaderm Magnolia Extract 98 | Magnolia Officinalis Bark | 1 |
| | | Extract | |
| | Mango butter, organic, | Mangifera Indica (Mango) | 3 |
| | deodorized | Seed Butter | |
| | Meadowfoam oil | Limnanthes Alba | 1 |
| | | (Meadowfoam) Seed Oil | |
| Water | | | |
| phase | | | |
| | Water, demin | Aqua | 57.4 |
| | Betaine | Betaine | 1 |
| | Symsitive 1609 | Pentylene Glycol + 4-t- | 1 |
| | | butylcyclohexanol | |
| | Pinolumin pwd | Pinus Cembra Wood Extract + | 2 |
| | | Maltodextrin + Aqua | |
| | Glycerin 99.5% Vegan | Glycerin | 2 |
| | Cinnamon extract | Cinnamomum Cassia Bark | 0.5 |
| | | Extract | |
| Thickener | | | |
| phase | | | |
| | Matrixyl synthe′6 | Glycerin + Water (Aqua) + | 2 |
| | | Hydroxypropyl Cyclodextrin + | |
| | | Palmitoyl Tripeptide-38 | |
| | Majestem | Glycerin + Leontopodium | 2 |
| | | Alpinum Callus Culture | |
| | | Extract + Xanthan Gum | |
| | UniqSens SFE System | Pectin + Xanthan Gum + | 0.3 |
| | | Carrageenan | |
| | Argirelox peptide solution | Aqua + Glycerin + Acetyl | 2 |
| | | Hexapeptide-8 + | |
| | | Pentapeptide-18 + Citric | |
| | | Acid + Caprylyl Glycol | |
| | Raya Hyaluron LMW | Sodium Hyaluronate | 0.6 |
| | Raya Hyaluron <10 Dalton | Sodium Hyaluronate | 0.4 |
| Perfume | | | |
| phase | | | |
| | Perfume Duigou Skin Uni 26 | Perfume | 0.8 |

### Example 6: Performance Cellulite Gel

| Phase/Phase | | | Amount as |
|---|---|---|---|
| temperature | Raw material | Inci | % |
| Water phase / | | | |
| 80° C. | | | |
| | Water, demin | Aqua | add 100 |
| | Symsave H | Hydroxyacetophenones | 0.5 |
| | Herbasec Guarana | Maltodextrin, Paullinia | 1 |
| | | Cupana Seed Extract | |
| | X50 Silhouette Powder | Coccoloba uviferaFruit | 0.005 |
| | | Extract, Lactic Acid/Glycolic | |
| | | Acid Copolymer, Polyvinyl | |
| | | Alcohol, Acetyl | |
| | | Cyclohexapeptide-34 | |
| | Butylene glycol 1,3 | Butylene glycol | 5 |
| | Snap-8 peptide solution C | Aqua, Caprylyl Glycol, Acetyl | 1 |
| | | Octapeptide-3 | |
| | Argirelox peptide solution | Aqua, Glycerin, Citric Acid, | 1 |
| | | Caprylyl Glycol, Acetyl | |
| | | Hexapeptide-8, | |
| | | Pentapeptide-18 | |
| | Caffeine Powder | Caffeine | 3 |
| | Sepitonic M3.0 | Aqua, Pentylene Glycol, | 1 |
| | | Magnesium Aspartate, Zinc | |
| | | Gluconate, Copper | |
| | | Gluconate | |
| | Glacier water from | Aqua, Cinnamomum Cassia | 10 |
| | Switzerland with cinnamon | Bark Extract | |
| | Nocturshape blue | Aqua, Propanediol, | 1 |
| | ingredient MB | Disodium Phosphate, | |
| | | Xanthan Gum, Sodium | |
| | | Phosphate, Glyceryl Caprylate, | |
| | | Plankton Extract | |
| | Eumulgin SG | Sodium Stearoyl Glutamate | 1 |
| | SymRelief 100 | Bisabolol, Zingiber Officinale | 1 |
| | | (Ginger) Root Extract | |
| Thickener | | | |
| phase / 25° C. | | | |
| | Vegenolin CF | Zea Mays (Corn) Oil, | 5 |
| | | Helianthus Annuus (Sunflower) | |
| | | Seed Wax, Lecithin, | |
| | | Sucrose Tetrastearate | |
| | | Triacetate, Glyceryl | |
| | | Undecylenate, Tocopherol | |
| | Chili Extrakt SoBluÖl | Helianthus Annuus (Sunflower) | 5 |
| | | Seed Oil, Capsicum | |
| | | Frutescens Fruit Extract | |
| | UniqSens SFE System | Pectin, Xanthan Gum, | 1.5 |
| | | Carrageenan | |
| Perfume | | | |
| phase /25° C. | | | |
| | Ethanol 96% (604 or 642) | Alcohol Denat. | 10 |
| | Blanova Benzylnicotinate | Benzyl Nicotinate | 0.25 |
| | DAB | | |
| | Perfume Duigou Skin 26 | Perfume | 0.6 |
| | Cosphaderm Magnolia | Magnolia | 0.75 |
| | Extract 98 | Officinalis Bark | |
| | | Extract | |
| | Cosphaderm VBE | Vanillyl Butyl Ether | 0.5 |

### Example 7: Cellulite Gel-Sensitive

| Phase/Phase | | | |
|---|---|---|---|
| temperature | Raw material | Inci | Amount as % |
| Water phase / | | | |
| 80° C. | | | |
| | Water, demin | Aqua | 50.145 |
| | Symsave H | Hydroxyacetophenones | 0.5 |
| | Butylene glycol 1,3 | Butylene glycol | 5 |
| | Pinolumin pwd | Maltodextrin, Aqua, Pinus | 1 |
| | | Cembra Wood Extract | |
| | Snap-8 peptide solution C | Aqua, Caprylyl Glycol, Acetyl | 1 |
| | | Octapeptide-3 | |
| | Argirelox peptide solution | Aqua, Glycerin, Citric Acid, | 1 |
| | | Caprylyl Glycol, Acetyl | |
| | | Hexapeptide-8, Pentapeptide- | |
| | | 18 | |
| | Caffeine Powder | Caffeine | 3 |
| | Sepitonic M3.0 | Aqua, Pentylene Glycol, | 1 |
| | | Magnesium Aspartate, Zinc | |
| | | Gluconate, Copper Gluconate | |
| | Glacier water from | Aqua, Cinnamomum Cassia | 10 |
| | Switzerland with cinnamon | Bark Extract | |
| | Nocturshape blue ingredient | Aqua, Propanediol, Disodium | 1 |
| | MB | Phosphate, Xanthan Gum, | |
| | | Sodium Phosphate, Glyceryl | |
| | | Caprylate, Plankton Extract | |
| | X50 Silhouette Powder | Coccoloba uviferaFruit | 0.005 |
| | | Extract, Lactic Acid/Glycolic | |
| | | Acid Copolymer, | |
| | | Polyvinyl Alcohol, Acetyl | |
| | | Cyclohexapeptide-34 | |
| | Eumulgin SG | Sodium Stearoyl Glutamate | 1 |
| | Herbasec Guarana | Maltodextrin, Paullinia | 1 |
| | | Cupana Seed Extract | |
| | SymRelief 100 | Bisabolol, Zingiber Officinale | 1 |
| | | (Ginger) Root Extract | |
| Thickener | | | |
| phase / 25° C. | | | |
| | Kahl SoftComplex 6422 | Rhus Verniciflua Peel Wax, | 5 |
| | | Simmondsia Chinensis | |
| | | (Jojoba) Seed Oil, Cetearyl | |
| | | Alcohol, Myristyl Alcohol, | |
| | | Caprylic/Capric Triglyceride, | |
| | | Copernicia Cerifera Cera, | |
| | | Helianthus Annuus | |
| | | (Sunflower) Seed Oil, | |
| | | Tocopherol | |
| | Raya Hyaluron SHMW | Sodium Hyaluronate | 0.25 |
| | Chili Extrakt SoBluÖl | Helianthus Annuus | 5 |
| | | (Sunflower) Seed Oil, | |
| | | Capsicum Frutescens Fruit | |
| | | Extract | |
| | UniqSens SFE System | Pectin, Xanthan Gum, | 1.5 |
| | | Carrageenan | |
| Perfume | | | |
| phase /25° C. | | | |

### Example 8: Luxury Face Cream

| | | |
|---|---|---|
| Ethanol 96% (604 or 642) | Alcohol Denat. | 10 |
| Perfume Duigou Skin 26 | Perfume | 0.6 |
| Cosphaderm Magnolia | Magnolia | 0.75 |
| Extract 98 | Officinalis Bark | |
| | Extract | |
| Cosphaderm VBE | Vanillyl Butyl Ether | 0.25 |

| Phase/Phase | | | Amount as |
|---|---|---|---|
| temperature | Raw material | Inci | % |
| Water phase / | | | |
| 80° C. | | | |
| | Water, demin | Aqua | 36.7 |
| | Pinolumin pwd | Maltodextrin, Aqua, Pinus | 1 |
| | | Cembra Wood Extract | |
| | Bio-Placenta | Aqua, Acetyl Glutamine, | 1 |
| | | Lecithin, sh-Oligopeptide-1, | |
| | | sh-Oligopeptide-2, sh- | |
| | | Polypeptide-1, sh- | |
| | | Polypeptide-11, sh-Polypeptide- | |
| | | 9, 1,2 Hexanediol, | |
| | | Bacillus/Folic Acid Ferment | |
| | | Extract, Butylene Glycol, | |
| | SYN-AKE | Glycerin, Aqua, Dipeptide | 2 |
| | | Diaminobutyroyl | |
| | Argirelox peptide solution | Aqua, Glycerin, Citric Acid, | 2 |
| | | Caprylyl Glycol, Acetyl | |
| | | Hexapeptide-8, | |
| | Glacier water from | Aqua, Cinnamomum Cassia | 10 |
| | Switzerland with cinnamon | Bark Extract | |
| | Witch hazel leaves Bio | Glycerin, Aqua, Hamamelis | 1 |
| | Extractive | Virginiana (Witch Hazel) Leaf | |
| | Snow Algae Powder | Maltodextrin, Aqua, | 2 |
| | | Coenochloris Signiensis | |
| | Betaine | Betaine | 2 |
| | Green Caviar Extract | Aqua, Butylene Glycol, | 5 |
| | | Caulerpa lentillifera extract | |
| Thickener | | | |
| phase /25° C. | | | |
| | Glycerin 99.5% Vegan | Glycerin, Aqua | 3 |
| | MVP Diamond Sirt | Maltodextrin, Acetyl sh- | 0.05 |
| | Powder | Pentapeptide-35, Diamond | |
| | Hydrolite 5 green | Pentylene Glycol | 5 |
| | Clearthix S | Cellulose Gum, Algin | 0.5 |
| Fat phase / | | | |
| 80° C. | | | |
| | Cetiol SB 45 | Butyrospermum Parkii | 0.6 |
| | | (Shea) Butter | |
| | Kahai Oil | Caryodendron Orinocense | 5 |
| | | Seed Oil | |
| | Coconut oil, virgin, organic | Cocos Nucifera (Coconut) | 2.5 |
| | | Oil | |
| | Nara Seed Oil | Acanthosicyos horridus Seed | 5 |
| | | Oil | |
| | Cosphaderm T-70 non | Tocopherol, Helianthus | 0.5 |
| | GMO | Annuus (Sunflower) Seed Oil | |
| | Lanette 22 | Behenyl Alcohol | 3 |
| | Prolix RB | Polyglyceryl-3 Rice Branate | 4.5 |
| | Macadamia nut oil, pressed | Macadamia Integrifolia Seed | 2 |
| | | Oil | |
| | Cold-pressed almond oil, | Prunus Amygdalus Dulcis | 5 |
| | organic | (Sweet Almond) Oil | |
| Perfume | | | |
| phase /25° C. | | | |
| | Perfume Duigou Skin Lux 26 | Perfume | 0.65 |

### Example 9: Super Luxury Cream

| Phase/Phase | | | Amount as |
|---|---|---|---|
| temperature | Raw material | Inci | % |
| Water | | | |
| phase / 80° C. | | | |
| | Water, demin | Aqua | ad 100 |
| | Pinolumin pwd | Maltodextrin, Aqua, Pinus | 2 |
| | | Cembra Wood Extract | |
| | Bio-Placenta | Aqua, Acetyl Glutamine, | 3 |
| | | Lecithin, sh-Oligopeptide-1, | |
| | | sh-Oligopeptide-2, sh- | |
| | | Polypeptide-1, sh- | |
| | | Polypeptide-11, sh- | |
| | | Ferment Extract, Butylene | |
| | | Glycol, Caprylyl Glycol, | |
| | | Sodium Hyaluronate | |
| | SYN-AKE | Glycerin, Aqua, Dipeptide | 3 |
| | | Diaminobutyroyl | |
| | | Benzylamide Diacetate | |
| | Argirelox peptide solution | Aqua, Glycerin, Citric Acid, | 5 |
| | | Caprylyl Glycol, Acetyl | |
| | | Hexapeptide-8, | |
| | | Pentapeptide-18 | |
| | Glacier water from | Aqua, Cinnamomum Cassia | 10 |
| | Switzerland with cinnamon | Bark Extract | |
| | Witch hazel leaves Bio | Glycerin, Aqua, Hamamelis | 1 |
| | Extractive | Virginiana (Witch Hazel) | |
| | | Leaf Extract | |
| | Snow Algae Powder | Maltodextrin, Aqua, | 3 |
| | | Coenochloris Signiensis | |
| | | Extract, Phospholipids | |
| | Betaine | Betaine | 2 |
| | Green Caviar Extract | Aqua, Butylene Glycol, | 5 |
| | | Caulerpa lentillifera extract | |
| Thickener | | | |
| phase / 25° C. | | | |
| | Glycerin 99.5% Vegan | Glycerin, Aqua | 3 |
| | MVP Diamond Sirt Powder | Maltodextrin, Acetyl sh- | 0.05 |
| | | Pentapeptide-35, Diamond | |
| | | Powder | |
| | Hydrolite 5 green | Pentylene Glycol | 5 |
| | Clearthix S | Cellulose Gum, Algin | 0.5 |
| Fat phase / | | | |
| 80° C. | | | |
| | Cetiol SB 45 | Butyrospermum Parkii (Shea) Butter | 0.6 |
| | Kahai Oil | Caryodendron Orinocense Seed Oil | 5 |
| | Coconut oil, virgin, | Cocos Nucifera (Coconut) Oil | 2.5 |
| | organic | | |
| | Nara Seed Oil | Acanthosicyos horridus Seed Oil | 5 |
| | Cosphaderm T-70 non | Tocopherol, Helianthus Annuus | 0.5 |
| | GMO | (Sunflower) Seed Oil | |
| | Lanette 22 | Behenyl Alcohol | 3 |
| | Prolix RB | Polyglyceryl-3 Rice Branate | 4.5 |
| | Macadamia nut oil, | Macadamia Integrifolia Seed Oil | 2 |
| | pressed | | |
| | Cold-pressed almond | Prunus Amygdalus Dulcis (Sweet | 5 |
| | oil, organic | Almond) Oil | |
| Perfume | | | |
| phase /25° C. | | | |
| | Perfume Duigou Skin | Perfume | 0.65 |
| | Lux 26 | | |

1-28. (canceled)

29. Topically applicable cosmetic or pharmaceutical preparation for skin care and for improving the condition of the skin, which contains a combination of at least 3 raw materials selected from
a) very low molecular weight hyaluronic acid with a molecular weight of less than 10 Daltons,
b) nara oil,
c) green caviar extract,
d) dipeptide diaminobutyroyl benzylamide diacetate,
e) coenochloris signiensis extract,
f) cinnamon extract
g) plankton extract,
h) acetyl cyclohexapeptide-34,
i) vanillyl butyl ether,
j) nicotinate or chili extract,
k) peptides,
l) hydroxystilbene or plant extracts containing hydroxystilbene and
m) glacier water

30. Preparation according to claim 29, wherein the combination of at least 3 raw materials is selected from
i. cinnamon extract, kahai oil, pinolumin and palmitoyl tripeptide-38,
ii. cinnamon extract, a derma membrane structure, pinolumin and acetyl octapeptide-3,
iii. cinnamon extract, argan oil butter, pinolumin and low molecular weight hyaluronic acid <10 Da,
iv. cinnamon extract, vitamin C ester, pinolumin, and active ingredient booster: pullulan,
v. cinnamon extract, acetyl tetrapeptide-17, collodial platinum acetyl octapeptide-3 and truffle extract,
vi. cinnamon extract, truffle extract, hormone-free sun protection factor system and acetyl octapeptide-3,
vii. nara oil, cinnamon extract, acetyl sh-pentapeptide-35, diamond powder and snow algae extract,
viii. plankton extract, cinnamon extract, acetyl cyclohexapeptide-34 and vanillyl butyl ether,
ix. acetyl octapeptide-3, cinnamon extract, acetyl cyclohexapeptide-34 and benzyl nicotinate

31. Preparation according to claim 29, wherein the peptides are selected from
acetyl tetrapeptide 17, in particular in form conjugated to colloidal gold or platinum, available from Infinitec Activos S.L;
acetyl tetrapeptide 17 conjugated to colloidal platinum—maltodextrin-based powder, available from Infinitec Activos S.L.;
acetyl octapeptide-3, available under the name SNAP-8™ from Lipotec S.A.U.;
the peptide available under the name Matrixyl® synthe'6® from Sederma;
acetyl hexapeptide-8 and pentapeptide-18, available under the name ARGRELOX™ from Lipotec S.A.U. as a peptide solution.

32. Preparation according to claim 29, wherein it has a total peptide content of 0.0001 to 5% by weight, in particular 0.01 to 4% by weight, preferably 0.1 to 3% by weight, particularly preferably 0.5 to 2% by weight.

33. Preparation according to claim 29, wherein the hydroxystilbenes are selected from 4′-hydroxystilbene, 2′,4′-dihydroxystilbene, 3′,4′-dihydroxystilbene, 4,4′-dihydroxystilbene, 3,5-dihydroxystilbene, 3-hydroxy-5-methoxystilbene, 2′,4′,4-trihydroxystilbene, 3′,4′,4-trihydroxystilbene, 2,4,4′-trihydroxystilbene, 3,4,4′-trihydroxystilbene, 3,4′,5-trihydroxystilbene, 2′,3,4-trihydroxystilbene, 2,3′,4-trihydroxystilbene, 2′,2,4′-trihydroxystilbene, 2,4,4′,5-tetrahydroxystilbene, 2′,3,4′,5-tetrahydroxystilbene, 2,2′,4,4′-tetrahydroxystilbene, 3,3′,4′,5-tetrahydroxystilbene, 2,3′,4,4′-tetrahydroxystilbene, 3,3′,4,4′-tetrahydroxystilbene, 3,3′,4′,5,5′-pentahydroxystilbene, 2,2′,4,4′,6-pentahydroxystilbene, 2,3′,4,4′,6-pentahydroxystilbene, 2,2′,4,4′,6,6′-hexahydroxystilbene, their alkyl ethers and/or their esters as well as trans-epsilon-viniferin, vitisin E and vitisin D and their alkyl ethers and/or their esters, in particular 3,5-dihydroxystilbene (pinosylvin).

34. Preparation according to claim 29, wherein it has a total hydroxystilbene content of 0.001 to 10% by weight, in particular 0.001 to 5% by weight, preferably 0.001 to 4% by weight, particularly preferably 0.005 to 1% by weight, extremely preferably 0.006 to 1% by weight.

35. Preparation according to claim 29, wherein the hydroxystilbene-containing plant extracts are selected from extracts from needles of the Pinophyta, in particular from the needles of the pine family, preferably the pine species Pinus ponderosa, Pinus albicaulis, Pinus aristata, Pinus attenuata, Pinus balfouriana, Pinus banksiana, Pinus bungeana, Pinus cembra, Pinus cembroides, Pinus clausa, Pinus contorta, Pinus coulteri, Pinus densiflora, Pinus echinata, Pinus edulis, Pinus elliottii, Pinus engelmannii, Pinus flexilis, Pinus glabra, Pinus heldreichii, Pinus jeffreyi, Pinus lambertiana, Pinus longaeva, Pinus massoniana, Pinus monophylla, Pinus monticola, Pinus mugo, Pinus muricata, Pinus nigra, Pinus palustris, Pinus parviflora, Pinus pungens, Pinus quadrifolia, Pinus radiata, Pinus resinosa, Pinus rigida, Pinus sabiniana, Pinus serotina, Pinus strobiformis, Pinus strobus, Pinus sylvestris, Pinus tabulaeformis, Pinus taeda, Pinus thunbergiana, Pinus torreyana, Pinus virginiana, Pinus yuannensis and Pinus washoensis, particularly preferably from Pinus cembra, most preferably the extract available under the name Pinolumin from Mibelle AG.

36. Preparation according to claim 29, wherein it has a total content of hydroxystilbene-containing plant extracts of 0.1 to 5% by weight, in particular 0.1 to 4% by weight, preferably 0.5 to 3% by weight, particularly preferably 1 to 2% by weight.

37. Preparation according to claim 29, wherein the glacier water is selected from
glacier water from Mont Blanc, available from Greentech GmbH, Starnberg under the name “Argentiere Ice Cap water”,
liposmomal encapsulated Swiss glacier water, available from Mibelle AG (Switzerland) under the name “Lipobelle Glacier”
glacier water from Switzerland, available from Botanica GmbH (Switzerland) and
glacier water from the Monte Rosa massif in Italy, available from Symrise, Holzminden under the name “Extrapone® Glacier Water GW”.
glacier water without listed preservatives

38. Preparation according to claim 29, wherein it has a total glacier water content of 1 to 20% by weight, in particular 5 to 15% by weight, preferably 7 to 13% by weight, particularly preferably 10% by weight.

39. Preparation according to claim 29, wherein it has a total content of very low molecular weight hyaluronic acid with a molecular weight of less than 10 daltons of 0.05 to 5% by weight, in particular 0.5 to 2% by weight, particularly preferably 0.75 to 1.25% by weight.

40. Preparation according to claim 29, wherein it has a total nara oil content of 2 to 10% by weight, in particular 3 to 8% by weight, particularly preferably 4 to 6% by weight.

41. Preparation according to claim 29, wherein it has a total green caviar extract content of 1 to 10% by weight, in particular 2.5 to 7.5% by weight, particularly preferably 3 to 5% by weight.

42. Preparation according to claim 29 for the treatment of cellulite and sagging skin, wherein it has a total plankton extract content of 0.05 to 5% by weight, in particular 0.1 to 2.5% by weight, particularly preferably 1 to 2% by weight, a total acetyl cyclohexapeptide-34 content of 0.00005 to 1% by weight, in particular 0.0005 to 0.5% by weight, particularly preferably 0.005 to 0.01% by weight, a total vanillyl butyl ether content of 0.05 to 5% by weight, in particular 0.1 to 2.5% by weight, particularly preferably 0.25 to 1.25% by weight, a total nicotinate content of 0.05 to 5% by weight, in particular 0.1 to 2.5% by weight, particularly preferably 0.25 to 1.25% by weight, and a total chili extract content of 0.05 to 10% by weight, in particular 0.1 to 5% by weight, particularly preferably 0.25 to 2.5% by weight.

43. Preparation according to claim 29, for the treatment of dry, wrinkled skin, wherein it has a total content of very low molecular weight hyaluronic acid with a molecular weight of less than 10 Daltons of 0.05 to 5% by weight, in particular 0.5 to 2% by weight, particularly preferably 0.75 to 1.25% by weight, a total nara oil content of 2 to 10% by weight, in particular 3 to 8% by weight, particularly preferably 4 to 6% by weight, a total green caviar extract of 1 to 10% by weight, in particular 2.5 to 7.5% by weight, particularly preferably 3 to 5% by weight, a total dipeptides diaminobutyroyl benzylamide diacetates content of 1 to 10% by weight, in particular 1 to 5% by weight, particularly preferably 2 to 3% by weight, a total coenochloris signiensis extract of 0.5 to 10% by weight, in particular 1 to 5% by weight, particularly preferably 2 to 3% by weight and a total cinnamon extract content of 0.05 to 2.5% by weight, in particular 0.5 to 1.5% by weight, particularly preferably 0.1 to 0.5% by weight.

44. Preparation according to claim 29, wherein it is in the form of an ointment or gel.

45. Preparation according to claim 29, wherein it improves the condition of cellulite.

46. Preparation according to claim 29, wherein it improves the condition of sagging skin.

47. Method for producing a preparation as described in claim 29 in the field of pharmacy or medicine for dermatological skin care and to improve the condition of the skin.

48. Method for producing a preparation as described in claim 29, wherein
a) very low molecular weight hyaluronic acid with a molecular weight of less than 10 Daltons,
b) nara oil,
c) green caviar extract,
d) dipeptide diaminobutyroyl benzylamide diacetate,
e) coenochloris signiensis extract,
f) cinnamon extract
g) plankton extract,
h) acetyl cyclohexapeptide-34,
i) vanillyl butyl ether,
j) nicotinate or chili extract,
k) peptides,
l) hydroxystilbene or plant extracts containing hydroxystilbene and
m) glacier water
as described for the preparations according to the invention, are mixed with cosmetically or pharmacologically suitable and tolerated carriers.
